(19) United States
(12) Patent Application Publication
(10) Pub. No.: US 2016/0123948 A1
(43) Pub. Date: May 5, 2016

(54) Method For Improving Animal Food Product Quality

(71) Applicant: Aytu Bioscience, Inc., Englewood, CO (US)

(72) Inventors: Raphael Bar-Or, Denver, CO (US); David Bar-Or, Englewood, CO (US); Leonard T. Rael, Centennial, CO (US)

(21) Appl. No.: 14/922,933

(22) Filed: Oct. 26, 2015

Related U.S. Application Data
(60) Provisional application No. 62/068,135, filed on Oct. 24, 2014.

(51) Int. Cl. G01N 33/12; G01N 33/08; G01N 33/04

(52) U.S. Cl. CPC G01N 33/12; G01N 33/04; G01N 33/08

(57) ABSTRACT

The invention relates to food animal care and husbandry. Disclosed is a novel method of evaluating the production characteristics of a food animal or group of food animals, is provided. More specifically, the invention relates to a method of determining the overall oxidative status of a sample taken from a food animal by measuring the oxidation-reduction potential (ORP) of the sample. The measured ORP value of the sample is then used to review the animal's environment and, if necessary, adjust the environment of the animal. In order to improve the production characteristics of food products from the animal. Also provided is a method of using the ORP value of a food product to determine production characteristics of the food product.

### TECHNICAL FIELD

The invention relates to a method of determining the overall oxidative status of a sample taken from a food animal by measuring the oxidation-reduction potential (ORP) of the sample. In particular, the invention relates to methods for evaluating the production characteristics of a food animal or group of food animals. The invention also relates to methods for improving the food characteristics of a food animal or group of food animals by measuring the ORP of these animals and adjusting the environment of the animals. Thus, the invention also relates to food animal care and husbandry.

### BACKGROUND OF INVENTION

Oxidative stress is caused by a higher production of reactive oxygen and reactive nitrogen species and/or a decrease in endogenous protective antioxidative capacity. In humans, oxidative stress has been related to various diseases and aging, and it has been found to occur in all types of critical illnesses. See, e.g., Veglia et al., Biomarkers, 11(6): 562-573 (2006); Roth et al., Current Opinion in Clinical Nutrition and Metabolic Care, 7:161-168 (2004); U.S. Pat. No. 5,290,519 and U.S. Patent Publication No. 2005/0142613. As with humans, the use of antioxidants as a dietary supplement to improve animal health is known (see PCT Patent Application Publication No. 2013/024067). U.S. Pat. No. 7,084,175 reports dietary feed compositions including fish oil, fish meal products and other marine animals containing omega-3 fatty acids for improving the reproductive performance of breeding populations of swine. PCT Patent Application Publication No. 2009/088879 discloses the use of dietary supplements such as antioxidants, trace minerals, organic acids, and essential amino acids for improving the health and production performance of animals. Although these references teach the value of dietary management to positively affect animal health, the test methods to assess the impact of the dietary changes are complex and time consuming. U.S. Patent Application Publication No. 2007/0162992 reports a method that combines spectroscopy and metabolomics, the study of the dynamic interplay of small molecule biomarkers, to provide a predictive tool for determining the ideal time for reproductive procedures in animals, including in vitro fertilization. Such a method is complex and is likely to face durability and reliability challenges in a real-world farming environment. PCT Patent Application Publication No. 20121173502 discloses a system for monitoring digestive efficiency within the rumen of ruminant animals comprising wireless transmitters equipped with, among other analyzers, pH sensors and redox sensors. Abnormal readings from the sensors alert the farmer to make dietary changes to create a better rumen environment, resulting in healthier, higher producing animals. These sensors are described as being placed in single animals or a sub-population of a farming herd (see www.kahneanimalhealth.com). These approaches lack a simple, fast and flexible method for reliably measuring the overall oxidative status of a diverse group of food animals, which require a wide variety of environmental conditions and diet, to produce a range of different food products

### SUMMARY OF THE INVENTION

One aspect of the invention is a method for evaluating a production characteristic of a food animal comprising measuring the oxidation-reduction potential (ORP) of a sample of the animal; and determining if the ORP is significantly different than a reference ORP value. Food animals suitable for use with the methods of the invention include a food animal for a food selected from the group consisting of meat, dairy, eggs and combinations thereof. In some aspects the food animal is selected from the group consisting of bovine, camelids, caprae, lagomorphs, ovis, swine, cervidae, birds, fish, crustaceans, mollusks, reptiles and combinations thereof. In some aspects, the food animal is selected from the group consisting of chickens, ducks, geese, pigeons, doves, pheasants, partridge, turkey, emu, swan, ostrich, bison, carabao, cattle, buffalo, yak, llama, camel, goat, rabbits, sheep, swine, fish, crustaceans, mollusks, reptiles and combinations thereof.

In some aspects of the invention, the production characteristic is selected from the group consisting of taste, flavor, freshness, appearance, color, color stability, fat content, lipid content, protein content, fatty acid content, cholesterol content, susceptibility to oxidation, amino acid content, collagen content, pigment content, fat color, glycogen level, smell, eating quality, juiciness, tenderness, aroma, texture, succulence, pH, R-value, sarcomere length, iron content, water holding capacity, firmness, marbling, cartilage maturity, bone maturity, dark cutting content, cutability, food animal growth rate, liveweight gain, feedlot gain, carcass weight, VIAscan® yield, feed efficiency, adult mortality rate, infection resistance, fertility, pregnancy failures, abortion rate after first trimester, number of young born dead or die within 24 hours, number of young die after 24 hours of age and weaning, calf-crop weaning percentage, eye health, teeth health, feet health, leg health and combinations thereof.

Additional production characteristics can be selected from the group consisting of eggs produced per animal per day, shell soundness, shell texture, shell shape, shell color, shell thickness, egg size, relative viscosity of the albumen, presence of foreign matter in the albumen, yolk firmness, yolk color, absence/presence of yolk defects, adult mortality rate, infection resistance and combinations thereof. In some aspects of the invention, the production characteristic is selected from the group consisting of bulk tank standard plate count, somatic cell count, lab pasteurized count, coliform count, preliminary incubation count, butterfat content, protein content, other solids content, rolling herd averages, rolling heard averages, first lactation metrics, greater than three lactation metrics, mortality rate, infection resistance, milk fever rate, ruminal acidocis rate, adult mortality rate, fertility, pregnancy failures, abortion rate after first trimester, number born dead or die within 24 hours, number die after 24 hours of age and weaning, calf-crop weaning percentage, eye health, teeth health, feet health, leg health, udder health, trace element status and combinations thereof.

In some aspects of the invention, the sample is selected from the group consisting of whole blood, blood serum, blood plasma, urine, feces, tissue, muscle, cartilage, bone, milk, whey, vaginal secretions, saliva, sweat, semen, amniotic fluid, cerebrospinal fluid, aqueous humour, vitreous humour, bile, chyle, chyme, gastric juice, lymph, pericardial fluid, peritoneal fluid, pleural fluid, pus, rheum, sputum, synovial fluid, sebum, egg yolk, egg albumen and combinations thereof.

In one aspect of the invention, a method of the invention comprises adjusting the environment to change the production characteristic of the food animal, wherein the food animal whose production characteristic to be changed is selected from the group consisting of the food animal, a progeny of the food animal, a cohort of the food animal, a next generation of the food animal and combinations thereof. The adjustment to the environment can be selected from the group consisting of administering a treatment to the food animal, changing the dietary regiment of the food animal, altering the living environment of the food animal, and combinations thereof.

In some aspects, adjusting the environment can comprise administering a treatment selected from the group consisting of medications, therapeutics, herbal treatments, ointments, salves, and combinations thereof. The treatment can be administered by a route selected from the group consisting of orally, intravenously, sublingually, rectally, vaginally, topically, subcutaneously, intramuscularly, and combinations thereof. In various aspects, the medication can be selected from the group consisting of hormones, antibiotics, antioxidants, anti-inflammatories, antivirals, and combinations thereof.

In some aspects of the invention, adjusting the environment comprises changing the dietary regiment selected from the group consisting of feed types, food ration delivery methods, quantity of feed provided, frequency of food provided, free or restricted access to feed, frequency of water provided, volume of water provided per unit time, the water quality, feed composition, daily allowance of protein, the dietary percentage of protein, type of grains provided, grain to straw ratio, presence fertilizers in feed, presence of pesticides in feed, and combinations thereof. Food types changed can be selected from the group consisting of hay, grains, protein, vitamin supplements, mineral supplements, scratch grains, straw, supplements, cracked corn, crushed oyster shells, crushed egg shells, fermented food scraps, flax seeds, grit, antioxidants, feeding limestone, salt, probiotics, crab meal, kelp, fish meal, cultured yeast, and combinations thereof.

In some aspects of the invention, adjusting the environment comprises altering the living environment selected from the group consisting of frequency of spiking male introduction, ratio of female to male animals, presence/absence of other species of food animals on farm, presence/absence of fencing, presence/absence of hard surface surrounding enclosures, presence/absence of tall brush surrounding enclosures, presence/absence of biosecurity protocols, presence/absence of dedicated service vehicles, number of animals per unit area, number of animal houses, age of animal houses, cleaning procedures, downtime between flocks, visitor access freqeuncy, spent- and dead-animal disposal methods, rodent and insect control measures, food product handling methods, lighting intensity, lighting duration, enclosure temperature, enclosure humidity, access to pasture, access to direct sunlight, ventilation, foggers, misters, sprinklers, cooling, stall surfaces, bedding, stall design, handling methods, loading methods, muster methods, occurrence and/or frequency of pen/mob mixing, method of dispatch, and combinations thereof.

In one aspect of the invention, the reference ORP value is determined based on factors selected from the group consisting of animal species, animal age, geographic location, any one of the above adjusting methods and combinations thereof.

In one aspect of the invention, the measuring, determining, and adjusting steps are performed on a single living food animal.

In one aspect of the invention, a method of the invention further comprises the step of dispatching the food animal, wherein the measuring and determining steps are performed on the sample of the butchered food animal.

In some aspect of the invention, the step of measuring the ORP is selected from the group consisting of measuring static ORP and capacity ORP. In some aspects, the reference ORP value is measured on the same food animal sample and the step of measuring the reference ORP value is performed in a similar manner. In some aspects, the step of measuring the ORP is performed at least once over the lifespan of the food animal. In some aspects, a method of the invention is performed on a population of food animals, wherein the population is selected from a group consisting of a flock, a herd, a cohort, a shoal, a brood, a clutch, a bed, a band, a litter, an army, a school, a hedge, a gang, a pod, a brace, a pack, a mob, a team, a stud, a troop, and combinations thereof.

One aspect of the present invention is a method of determining a characteristic of a food from a food animal, comprising measuring one or more ORP values of the food product; and determining the characteristic from the measured ORP value. The characteristic can be selected from the group consisting of taste, flavor, freshness, appearance, color, color stability, fat content, lipid content, protein content, fatty acid content, cholesterol content, susceptibility to oxidation, amino acid content, collagen content, pigment content, fat color, glycogen level, smell, eating quality, juiciness, tenderness, aroma, texture, succulence, pH, R-value, sarcomere length, iron content, water holding capacity, firmness, marbling, cartilage maturity, bone maturity, dark cutting content, cutability, shell soundness, shell texture, shell shape, shell color, shell thickness, egg size, relative viscosity of the albumen, presence of foreign matter in the albumen, yolk firmness, yolk color, absence/presence of yolk defects, bulk tank standard plate count (SPC), somatic cell count (SCC), lab pasteurized count (LPC), coliform count, preliminary incubation count (PIC), butterfat content, protein content, other solids content, rolling herd averages (pounds milk produced, % butterfat, % protein), rolling heard averages (pounds milk produced, % fat, % protein), and combinations thereof.

One aspect of the present invention is a method of determining a characteristic of meat, comprising measuring one or more ORP values of a sample from the meat; and determining color stability from the measured ORP value. In some aspects, the characteristic can be selected from the group consisting of taste, flavor, freshness, appearance, color, color stability, fat content, lipid content, protein content, fatty acid content, cholesterol content, susceptibility to oxidation, amino acid content, collagen content, pigment content, fat color, glycogen level, smell, eating quality, juiciness, tenderness, aroma, texture, succulence, pH, R-value, sarcomere length, iron content, water holding capacity, firmness, marbling, cartilage maturity, bone maturity, dark cutting content, and cutability.

One aspect of the present invention is a method of optimizing and improving the health and/or food characteristics (production characteristics) of a food animal of a food animal, or a group of food animals, comprising measuring one or more ORP values of a food product from the food animal, or a food product from at least one animal in the group of animals; determining at least one food characteristic from the measured ORP value; and, based on the measured ORP value, adjusting the environment of a second food animal, or group of animals, to change the production characteristic of the second food animal, or group of animals, to be different from the previous production characteristic of the food animal from which the sample was taken, wherein the second food animal, or group of animals, is selected from the group consisting of the food animal, a progeny of the food animal, a cohort of the food animal, a next generation of the food animal and combinations thereof. In some aspects, the food characteristic being optimized or improved can be selected from the group consisting of taste, flavor, freshness, appearance, color, color stability, fat content, lipid content, protein content, fatty acid content, cholesterol content, susceptibility to oxidation, amino acid content, collagen content, pigment content, fat color, glycogen level, smell, eating quality, juiciness, tenderness, aroma, texture, succulence, pH, R-value, sarcomere length, iron content, water holding capacity, firmness, marbling, cartilage maturity, bone maturity, dark cutting content, cutability, food animal growth rate, liveweight gain, feedlot gain, carcass weight, VIAscan® yield, feed efficiency, adult mortality rate, infection resistance, fertility, pregnancy failures, abortion rate after first trimester, number of young born dead or die within 24 hours, number of young die after 24 hours of age and weaning, calf-crop weaning percentage, eye health, teeth health, feet health, leg health, eggs produced per animal per day, shell soundness, shell texture, shell shape, shell color, shell thickness, egg size, relative viscosity of the albumen, presence of foreign matter in the albumen, yolk firmness, yolk color, absence/presence of yolk defects. In some aspects, the step of adjusting the environment comprises an adjustment selected for the group consisting of administering a treatment to the second food animal, changing the dietary regime of the second food animal, altering the living environment of the second food animal, and combinations thereof.

### BRIEF DESCRIPTION OF THE FIGURES

FIG. 1 depicts components of a system 100 for measuring the oxidation-reduction potential (ORP) value.

FIG. 2 illustrates additional components and features of a readout device.

FIG. 3 depicts details of the analog front end 220 and of the electrical circuit associated with the test strip 108 of a system of the invention.

FIG. 4 depicts aspects of a test strip of a system of the invention.

FIG. 5 is a flowchart illustrating aspects of the operation of a system for determining ORP.

FIG. 6 depicts current supplied by a readout device to an interconnected test strip over time.

FIG. 7 The relationship of myoglobin states with meat color

FIG. 8 Change in the oxidation of lipids from a meat sample over time. Samples of beef were processed to obtain lipids, the lipids incorporated into phosphotidylcholine liposomes, and oxidation of the meat lipid measured over time.

FIG. 9 Change in ORP value of lipids from a meat sample over time. Samples of beef were processed to obtain lipids, the lipids incorporated into phosphotidylcholine liposomes, and ORP values of the liposomes measured over time.

FIG. 10 Comparison of change in lipid oxidation and antioxidant capacity of a meat sample over time. Samples of beef were processed to obtain lipids, the lipids incorporated into phosphotidylcholine liposomes, and the lipid oxidation value and the antioxidant capacity of the liposomes measured over time. A) change in the amount of oxidized lipid over time. B) Change in the antioxidant capacity of the same samples from panel A.

FIG. 11 Comparison of ORP value of meat with pH and meat color. Steaks were aged for 21 days at different pH values. Samples of each steak were taken and processed as described in Example 2. ORP values were measured and correlated with meat color. A) ORP value of meat samples aged at normal ph (5.6) or high pH 6.4; Dark Cutter). B) Steaks aged at pH 6.4 (Top) of ph 5.6 (Bottom).

FIG. 12 Effect of packaging on ORP values. Meat was packaged either in PVC or in HiOx (HiOx=80% oxygen+20% carbon dioxide). Samples were taken and ORP values measured.

FIG. 13 Effect of time and ozone on ORP values in meat. Meat was incubated for various times, at various temperatures, under normal room air or in the presence of ozone. Samples were taken and OPR measured.

### DESCRIPTION OF EMBODIMENTS

The following detailed description illustrates the invention by way of example and not by way of limitation. This description enables one skilled in the art to make and use the invention.

References in the specification to “one embodiment”, “an embodiment”, “an example embodiment”, etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described.

As used herein, “food animal” refers to bovine, camelids, caprae (goats), lagomorphs (rabbits), ovis (sheep), swine, cervidae (deer), birds, fish, crustaceans, mollusks, and reptiles.

Examples of birds include, but are not limited to, chickens, ducks, geese, pigeons, doves, pheasants, partridge, turkey, emu, swan, and ostrich. Other examples of food animals include, but are not limited to, bison, carabao, cattle, buffalo, yak, and llama.

As used herein, “sample” taken from a food animal for ORP testing may be any suitable fluid including, but not limited to, a blood sample (e.g., whole blood, serum or plasma), urine, saliva, cerebrospinal fluid, tears, semen, vaginal secretions, amniotic fluid and cord blood. Other fluids include but are not limited to, milk, whey, aqueous humour, vitreous humour, bile, chile, chyme, gastric juice, lymph, pericardial fluid, peritoneal fluid, pleural fluid, pus, rheum, sputum, synovial fluid, sebum, egg yolk, and egg albumin. The “sample” taken from a food animal for ORP testing may be any suitable solid or tissue including, but not limited to, epithelial tissue, connective tissue, muscle tissue and nervous tissue. The “sample” taken may be the food product itself. The “sample” may include bone, cartilage, and organs. Also, lavages, tissue homogenates and cell lysates can be utilized and, as used herein, “sample” includes such preparations.

As used herein, “food product” refers to products obtained from a food animal and includes, but is not limited to, meat, egg, dairy, blood, bone, stock, gelatin, lard, oil, and whey.

Embodiments of the present invention provide systems and methods for measuring oxidation-reduction potential (ORP) characteristics (i.e., static oxidation-reduction potential (sORP) and/or oxidation-reduction capacity (cORP)) of a sample that are suitable for rapid, robust, and routine testing in a farm environment, or other animal raising, rearing, or caring environment, and methods of using the system to evaluate or monitor the status of food animals. The system generally includes a test strip and a readout device. More particularly, embodiments of the present invention can determine the ORP characteristics of a sample of a food animal in a convenient and timely manner. A biological sample of a food animal that can be used in the method of the invention can be any fluid, as described above.

The test strip generally includes a substrate, a reference cell, a counter electrode, a working electrode, a reference electrode, and a sample chamber. In general, by placing a sample in the sample chamber, an electrical connection is established between the reference cell, the counter electrode, the working electrode, and the reference electrode. The test strip can then be connected to a readout device, for the determination of a static ORP value and an ORP capacity value.

The readout device generally includes contacts to electrically interconnect the readout device to the various electrodes included in the test strip. In accordance with embodiments of the present disclosure, the readout device includes an analog front end. The analog front end generally functions to provide a controlled current that can be sent across the sample in the sample chamber through an electrical connection to the counter electrode and the working electrode. In addition, the analog front end is operable to generate a voltage signal that represents the potential difference between the reference electrode and the working electrode. An analog to digital (ADC) converter is provided to convert the voltage signal representing the reference electrode to working electrode potential difference to a digital signal. A digital to analog converter (DAC) is provided to convert a digital control signal to analog signals in connection with the provision of the controlled current to the test strip. A controller interfaces with the ADC and the DAC. Moreover, the controller can include or comprise a processor that implements programming code controlling various functions of the readout device, including but not limited to controlling the current supply to the test strip, and processing the potential difference measurement signal. The controller can operate in association with memory. In addition, the readout device includes a user interface, and a power supply.

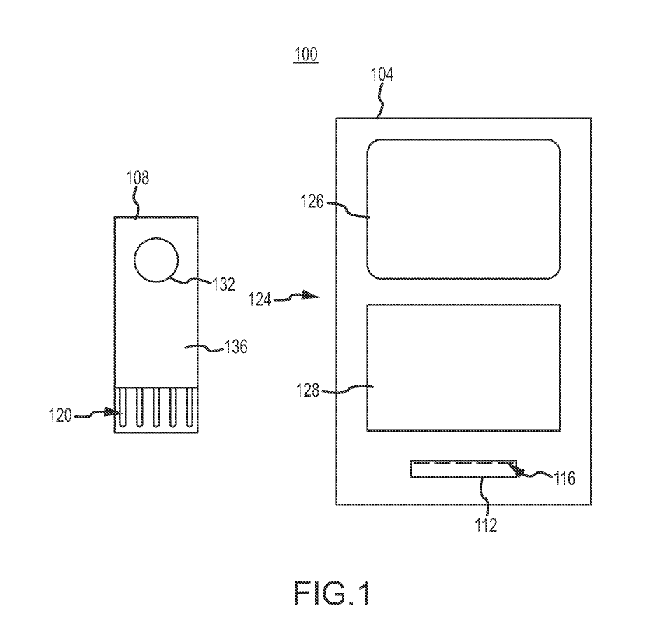

FIG. 1 depicts components of a system 100 for measuring the oxidation-reduction potential (ORP) value, including but not limited to the static oxidation-reduction value (sORP) and/or the oxidation-reduction capacity value (cORP), of a sample in accordance with embodiments of the present disclosure. As used herein, the sORP is a measured potential difference or voltage across a sample such as a measured potential difference or voltage across a sample placed in a test strip that includes a reference cell as described herein. The cORP as used herein is a measure of the quantity of charge provided to a sample over a defined period such as can be measured in a test strip as described herein. Accordingly, the cORP can be viewed as the capacity of a sample to absorb an electrical charge supplied as a current over some defined period. In some embodiments of the present invention, the sample may be a fluid. For example, the period can be defined by the initiation of current supply to a sample and an endpoint such as an inflection point or by a first and a second inflection point. In general, the system 100 includes a readout device 104, which can implement a galvanometer, and a test strip 108. The readout device 104 includes a connector or readout aperture 112 for electrically interconnecting readout contacts 116 of the readout device 104 to electrode contacts 120 provided as part of the test strip 108. The readout device can also incorporate a user interface 124, which can include a user output 126, such as a display, and a user input 128, such as a keypad. In accordance with still other embodiments, the user interface 124 can comprise an integrated component, such as a touch screen interface. In addition to providing contacts 120 for interconnecting the test strip 108 to the readout device 104, the test strip 108 includes a sample chamber aperture 132 formed in a test strip overlay 136, to receive a sample in connection with the determination of an ORP value of that sample.

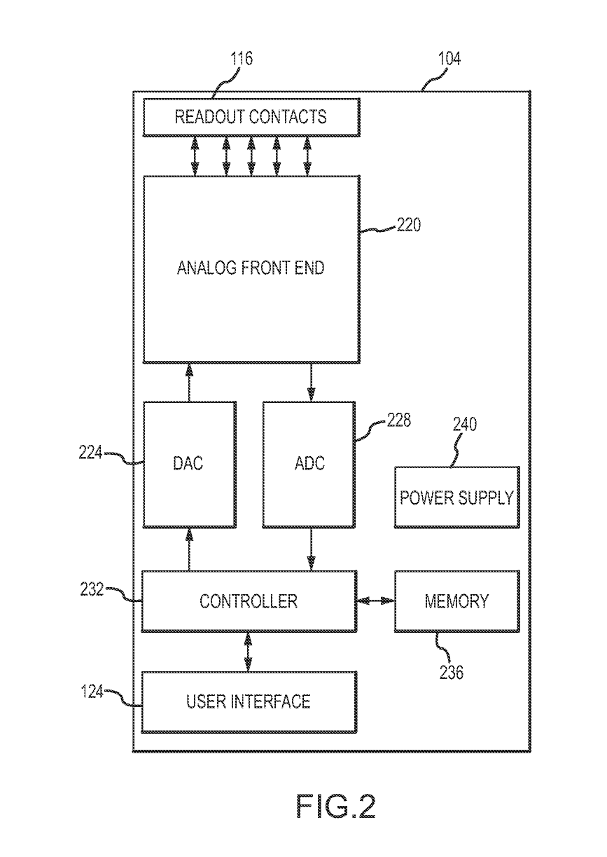

FIG. 2 illustrates additional components and features of a readout device 104 in accordance with embodiments of the present disclosure. As shown, the readout contacts 116 are interconnected to an analog front end 220. As described in greater detail elsewhere herein, the analog front end 220 generally functions to provide a controlled current that is passed between a counter electrode and working electrode of the test strip 108. In addition, the analog front end 220 functions to provide a voltage signal representing a potential difference between a reference electrode and the working electrode of the test strip 108. In accordance with still further embodiments, the analog front end 220 can include a strip detect circuit, to provide a signal indicating the interconnection of a test strip 108 to the readout device 104.

The analog front end 220 generally receives control signals from a digital to analog (DAC) converter 224. Signals output by the analog front end 220 are generally provided to an analog to digital converter (ADC) 228. The DAC 224 and ADC 228 are in turn connected to a controller 232. The controller 232 may comprise a processor that is operable to execute instructions stored in memory as part of the controller 232, or as a separate memory device 236. For example, the processor, executing instructions stored in memory 236, can implement a process according to which the current supplied to the test strip 108 is controlled. In addition, the controller 232 can execute instructions stored in memory 236 to record the quantity of current supplied to the test strip 108, to detect an inflection point in the voltage potential between electrodes of the test strip 108, and to calculate an ORP capacity. The memory 236 can also function as storage for data, including but not limited to intermediate and/or final ORP values. The controller 232 can comprise a general purpose programmable processor or controller or a specially configured application integrated circuit (ASIC).

The user interface 124 generally operates to provide user input to the controller 232. In addition, the user interface 124 can operate to display information to a user, including but not limited to the status of the readout device 104 or of the system 100 generally, a sORP value, and a cORP value.

The readout device 104 also generally includes a power supply 240. Although not shown in the figure, the power supply 240 is generally interconnected to power consuming devices via a power supply bus. The power supply 240 may be associated with a battery or other energy storage device, and/or line power.

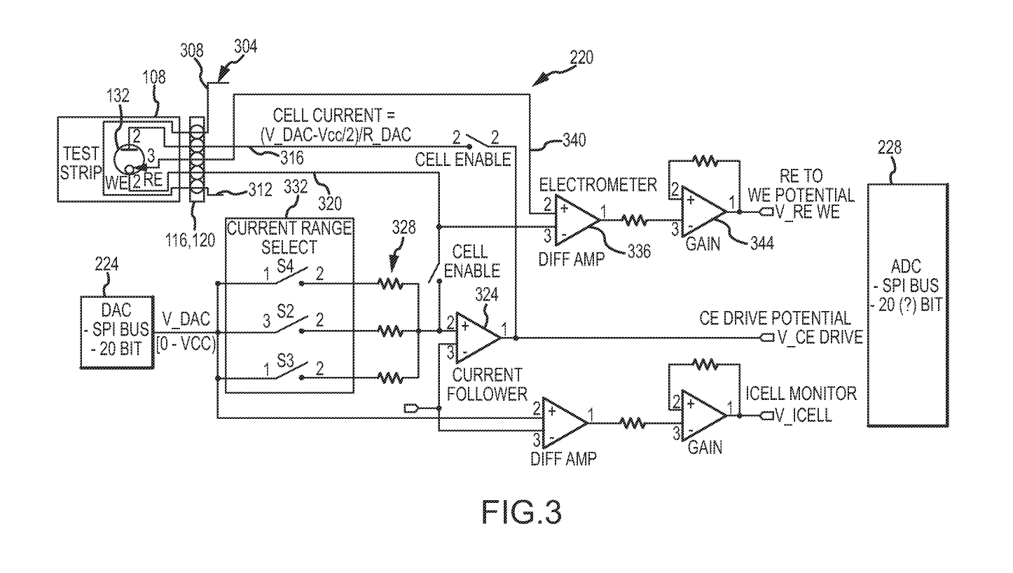

With reference now to FIG. 3, additional features of a system 100 in accordance with embodiments of the present disclosure are depicted. More particularly, details of the analog front end 220 and of the electrical circuit associated with the test strip 108 are depicted. As shown, the readout contacts 116 interconnect to the electrode leads or contacts 120, to electrically connect the analog front end 220 to the test strip 108. In the illustrated embodiment, the analog front end 220 includes a test strip sense circuit 304. The test strip sense circuit 304 includes a test strip detection supply lead 308 and a test strip detection input lead 312. In general, when a suitable test strip 108 is operatively connected to the readout device 104, continuity between the test strip detect supply lead 308 and the test strip detection input lead 312 is established, allowing a test strip detect signal indicating that a test strip 108 is present to be passed between the supply 308 and the input 312 leads. Moreover, a test strip 108 can incorporate a resistor or other component to modify the test strip detect signal, to indicate to the readout device 104 characteristics of the particular test strip 108 that has been interconnected to the readout device 104, such as the voltage value of a reference cell incorporated into the test strip 108. In response to sensing the presence of a test strip 108, the readout device 104 can operate to provide an interrogation signal in the form of a controlled current to the test strip 108.

The current is provided by the readout device 104 to the sample chamber 132 of the test strip 108 via a counter electrode lead 316 and a working electrode lead 320. More particularly, the current may be supplied to the counter electrode lead 316 from the output of a current follower 324, while the working electrode 320 can be provided as an input to that current follower 324. In addition, a set of current range select resistors 328 and associated switches 332 can be controlled by the DAC 224, as directed by the controller 232, for example depending on the characteristics of the interconnected test strip 108. In addition, the DAC 224, as directed by the controller 232, can control the input to the current follower 324 to in turn control the amount of current supplied to the test strip 108 by the current electrode lead 316. The DAC 224, as directed by the controller 232, can also operate various switches and/or amplifiers to control the operating mode of the analog front end 220.

The analog front end 220 additionally includes an electrometer 336 that receives a first input signal from a reference electrode lead 340 and a second input signal from the working electrode lead 320. The output from the electrometer 336 generally represents the potential difference between the reference electrode lead 340 and the working electrode lead 320. The signal output by the electrometer 336 can be amplified in a gain circuit 344, and output to the ADC 228.

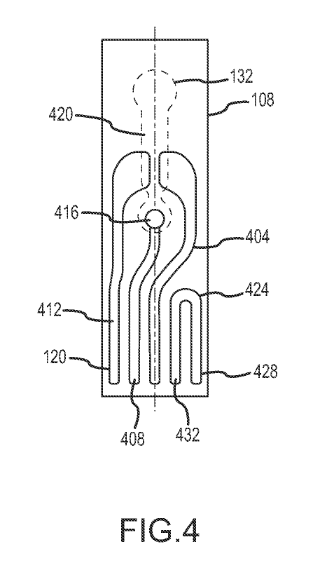

FIG. 4 depicts aspects of a test strip 108 in accordance with embodiments of the present invention. More particularly, the view presented by FIG. 4 shows the test strip 108 with the test strip overlay 136 removed. In general, the test strip 108 includes a working electrode 404, a reference electrode 408, and a counter electrode 412. In addition, the test strip 108 includes a reference cell 416. By placing a sample within a sample chamber region 420, the working electrode 404, the reference electrode 408, the counter electrode 412, and the reference cell 416 are placed in electrical contact with one another. Moreover, by placing the electrode contacts 120 corresponding to the counter electrode 412, the working electrode 404 and the reference electrode 408 in contact with the readout contacts 116 corresponding to the counter electrode lead 316, the working electrode lead 320, and the reference electrode lead 340 respectively, the test strip 108 is operatively connected to the readout device 104. Accordingly, a supply current provided to the test strip 104 can be sent across the sample, between the counter electrode 412 and the working electrode 404 by the readout device 104. Moreover, the potential difference between the reference electrode 408 and the working electrode 404 can be sensed by the readout device 104. In accordance with further embodiments of the present disclosure, the test strip 108 can include a test strip detect circuit 424, that includes an input 428 and an output 432. The test strip detect circuit 424 can, in addition to the input 428 and the output 432, include a resistor or other component for modifying a test strip sense signal provided by the readout device 104, to indicate to the readout device 104, an identification of the test strip 108.

To measure the cORP or antioxidant reserve, the sample is titrated with a linearly increasing oxidizing current between a counter and working electrode to exhaust the relevant antioxidants at the working electrode while monitoring the voltage between the working and reference electrodes. The result is a time versus voltage curve and a time versus current curve. The time versus voltage curve is used to find an inflection point where the voltage is changing the fastest (antioxidants are exhausted so system tries to find a new equilibrium). The time at maximum velocity is referred to as the transition time. The capacity or cORP is then the integral of the current profile from the beginning to the transition time with units of uC.

Calculation of the transition time may be accomplished several ways including noise filtration, curve fitting and standard numerical differentiation techniques. Usually the unfiltered numerical derivative is noisy, making finding maxima difficult or unreliable. To that end, one technique is to curve fit the time versus voltage profile with a polynomial (5th-7th order is usually sufficient) and directly differentiating the resulting polynomial analytically. This approach has the advantage of very smooth derivatives making the determination of the transition time robust as long as the fit is good.

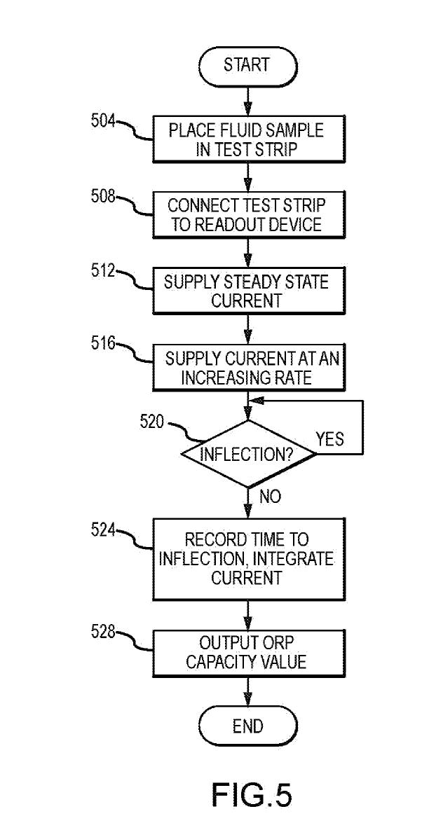

FIG. 5 is a flowchart illustrating aspects of the operation of a system 100 for determining the ORP, including but not limited to the cORP, of a sample in accordance with embodiments of the present invention. In general, the method includes obtaining a sample and placing the sample in the sample chamber 420 of a test strip 108 (step 504). In some embodiments of the present invention, the sample may be a fluid. At step 508, the test strip 108 is connected to the readout device 104 (step 508). In general, while the readout device 104 is in an on or standby mode, an electrical signal may be output by the test strip detection output lead 308. By connecting a suitable test strip 108 to a readout device 104, continuity between the test strip detect output lead 308 and the test strip detect input lead 312 is established. In addition, the signal received at the test strip detect input lead 312 can provide an indication of characteristics of the test strip 108, which can in turn be used to control aspects (e.g., a current range) of a current supplied to the test strip 108. Such characteristics can include but are not limited to the type and composition of the test strip electrodes 404, 408 and 412, and the potential of the reference cell 416.

At step 512, a current can be supplied by the readout device 104 to the counter electrode 412 of the test strip 108. More particularly, a current can be passed between the counter electrode 412 and the working electrode 404 by the counter electrode lead 316 and the working electrode lead 320. In accordance with embodiments of the present disclosure, the current that is supplied to the test strip 108 is controlled by the controller 232 of the readout device 104. More particularly, the current can be provided for at least a first segment of time at a selected, steady state level. The first segment of time can be a fixed time period. Alternatively, the first segment of time can expire once a determination has been made that the potential difference sensed by the readout device 104 between the reference electrode 408 and the working electrode 404 has a rate of change that is less than some selected amount. In accordance with still other embodiments, a combination of parameters may be applied to determine the time period over which the current is supplied at a steady state. Moreover, in accordance with other embodiments, no current is supplied during the first period of time (i.e. the supplied current during the first segment of time is zero). As can be appreciated by one of skill in the art after consideration of the present disclosure, while no current is supplied and while the rate of change of that potential difference is zero or less than some selected amount, the potential difference measured by the readout device 104 between the reference electrode 408 and the working electrode 404 is equal to the sORP of the sample.

After the first segment of time has expired, the current can be supplied at an increasing rate (step 516). For example, the amount can be increased linearly, as a step function, exponentially, according to a combination of different profiles, or in any other fashion. For instance, the current can be increased linearly from 0 amps at a specified rate until an endpoint is reached. As another example, the amount can be stepped from 0 amps to some non-zero value, and that non-zero value can be provided at a steady rate for some period of time, or can be provided at an increasing rate according to some function. At step 520, a determination can be made as to whether an inflection point in the potential difference monitored between the reference electrode 408 and the working electrode 404 has been detected. More particularly, the reference electrode lead 340 and the working electrode lead 320 connect the reference electrode 408 and the working electrode 404 respectively to the electrometer 336, which outputs a signal representing the potential difference between the reference 408 and the working 404 electrodes. The analog to digital converter 228 then converts the signal representing the potential difference between the reference 408 and working 404 electrodes to a digital signal that is provided to the controller 232. If an inflection point has been detected, the readout device 104, and in particular the controller 232, can record the time from which current was first supplied to the time at which the inflection point is reached. In addition, the controller 232 can integrate the current signal to determine an amount of charge that has been supplied to the sample up to the time at which the inflection point is reached (step 524). In accordance with embodiments of the present disclosure, a first inflection point (e.g., a point at which the voltage measured across a sample while a current is being supplied is at a local maximum rate of change) is used as the point at which integration of that amount is stopped. However, multiple inflection points can be observed in the measured voltage. Accordingly, rather than using the first observed inflection point as the end point for integration, a subsequent inflection point can be used. As yet another example, a time determined with reference to multiple inflection points, such as a midpoint between two observed inflection points or an average time of multiple observed inflection points can be used as the end point of the integration for purposes of determining the cORP of a sample. At step 528, the determined quantity of charge or a value derived from the determined quantity of charge can be output to a user as an ORP capacity (cORP) value for the sample, for example through the output device 128 facility of a user interface 124 provided as part of or interconnected to a readout device 104. For example, the cORP value can be defined as one over the quantity of charge. The process can then end. In some embodiments of the present invention, the sample may be a fluid.

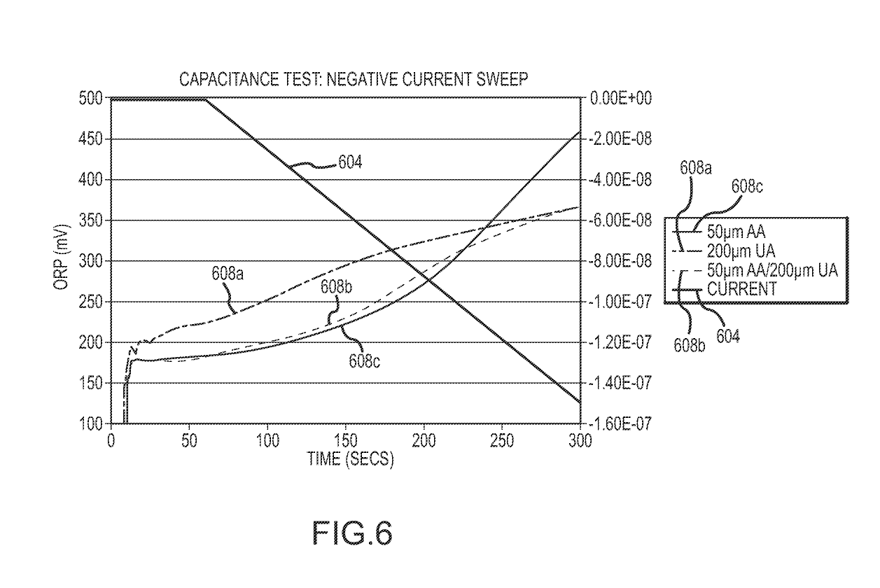

FIG. 6 depicts the current, shown as line 604, supplied by a readout device 104 to an interconnected test strip 108 over time. In addition, sample measured potential difference values 608a-c for different exemplary samples are depicted. As can be understood by one of skill in the art after consideration of the present disclosure, although three potential difference values 608 are shown, a current 604 is provided to only one sample during determination of an ORP value. As can also be appreciated by one of skill in the art after consideration of the present disclosure, the ramped portion of the current 604 is shown sloping in a downward direction, because it depicts an oxidizing current. In addition, it can be appreciated that the area between the current curve 604 and a current value of zero for a selected period of time represents a quantity of charge provided to a sample held in a test strip 108. Accordingly, this quantity of charge can be used to provide a measurement of the ORP capacity (cORP) of the sample. Moreover, the voltage curves 608 represent a static ORP (sORP) value of a respective sample at different points in time. The area under the current curve 604 (which is above the curve 604, between that curve and a current of zero in FIG. 6) that is used to determine the cORP can have a start point at a first point in time and an end point at a second point in time. As an example, the start point for integration of the current 604 can be selected as a point at which the observed sORP signal or reading has stabilized. For instance, in the example of FIG. 6, the potential difference values have stabilized after about 50 seconds have elapsed. Moreover, in this example no current is being supplied to the sample by the readout device 104 during the first segment of time leading up to the start point at which current is supplied. That start point can also correspond to the time at which the current 604 begins to be applied at an increasing rate. In accordance with embodiments of the present disclosure, where a curve 608 reaches an inflection point, for example the point at which the rate of change in the measured potential difference is at a maximum (i.e., a point of maximum slope), the integration of the current signal 604 is stopped. For example, looking at curve 608b, an inflection point can be seen at about 200 seconds, and integration of the current 604 can thus be performed during the period beginning at 50 seconds and ending at 2,000 seconds. Alternatively, the integration of the current signal 604 can be stopped after some predetermined period of time. As yet another alternative, the integration of the current signal 604 can be stopped at the earlier of the observation of an inflection point or the expiration of a predetermined period of time.

As can be appreciated by one of skill in the art after consideration of the present disclosure, the measurement of the sORP value can be in units of Volts, and the integration of the current signal or value 604 therefore gives a value representing a quantity of charge in Coulombs. cORP values, as a measure of a quantity of charge, is expressed herein as one over the quantity of charge in Coulombs. In particular, by taking the inverse of the observed quantity of charge, a more normal distribution is obtained, facilitating the application of parametric statistics to observed ORP values. As used herein, the terms ORP capacity, inverse capacity levels, inverse capacity ORP or ICL are all equivalent to cORP as defined above. It will be appreciated that expression of cORP as one over a quantity of charge encompasses alternative equivalent expressions.

As noted above, higher than normal values of sORP are indicative of oxidative stress and are considered to be a negative indication for the food animal being evaluated. cORP is a measure of an animal's capacity to withstand oxidative insult. Thus, it is a positive indication for an animal to have a normal or higher capacity to withstand oxidative insult. Since cORP is defined as the inverse of the quantity of charge to reach a voltage inflection point, a higher cORP value is indicative of a lesser capacity to withstand oxidative insult, and likewise, a lower cORP value is indicative of a greater capacity to withstand oxidative insult.

The present invention includes embodiments for monitoring or evaluating the health of food animals having a variety of conditions by determining the ORP characteristics of a biological sample of the food animal. Typically, the ORP characteristics of the food animal are compared to an ORP characteristic reference value or values that are relevant to that animal. As used herein, a reference value can be an ORP characteristic of the food animal from a time when the animal did not have the condition in question (i.e., when the animal was healthy) or from an earlier time period when the animal had the condition in question (for purposes of monitoring or evaluating the condition or treatment thereof). Such reference values are referred to as self reference values. For example, reference values can also include initial, maximum and ending reference values, such as when ORP characteristics are evaluated over a time frame such as when a food animal is being introduced to a new environmental condition or diet (initial), during a stay in a new environment, e.g. shelter in a climate controlled barn (maximum), and at a time when a food animal is transferred back to a typical environmental condition, e.g. released back to a field, or placed back on a standard diet (ending). Alternatively, a reference value can be an ORP characteristic of a relevant healthy population, herd, flock, etc. (e.g., a population that is matched in one or more characteristics of species, age, sex, geographic location, etc.). Such reference values are referred to as normal reference values. Further, a reference value can be an ORP characteristic of a relevant population similarly situated as the food animal (e.g., a population having the same or similar condition as the animal for which the animal is being treated and preferably, one that is also matched in one or more characteristics of species, age, sex, geographic location, etc.). Such a reference value is referred to as a condition specific reference value. For example, a condition specific bovine reference value can be a coccidiosisreference value, a drought condition reference value, or a non-drought condition reference value.

As used herein, a subject is any individual for whom a biological sample is being tested for an ORP characteristic. The term subject can include a food animal if the subject is an animal being farmed, grown, or raised to produce animal products, in a farm environment or any other relevant animal husbandry environment, and cared for by a human being. The terms subject and animal can refer to any animal, including companion animals (e.g., cats, dogs, horses, etc.) and food animals (i.e., animals kept for food purposes such as cows, goats, chickens, etc.). Preferred subjects include mammals and most preferably food animals.

In various embodiments of the invention, the ORP characteristics of a biological sample of a subject are measured. The measurement of the ORP characteristics of a biological sample can be done at multiple time points. The frequency of such measurements will depend on the condition being evaluated, the species of animal being raised, the food product produced, the sample size of the animals being tested, the farm conditions and environment, and the season, among other things. For example, urgent conditions such as during a disease outbreak in a herd can employ more frequent testing of an individual animal or multiple animals. In contrast, establishing long-term reference conditions such as for a flock of healthy sheep may employ longer term testing intervals. As such, for example, testing can be done every 30 minutes, hour, 2 hours, 3 hours, 4 hours, 5 hours, 6 hours, 12 hours, 18 hours, or day for urgent medical conditions or for food animal species that are frequently producing food products over shorter time intervals. Alternatively, testing can be done every day, 2 days, 3 days, 4 days, 5 days, 6 days, week, 2 weeks, 3 weeks, month, 2 months, 3 months, 4 months, 5 months, 6 months, 7 months, 8 months, 9 months, 10 months, 11 months, or year for food animals with long life spans, for example, dairy cattle.

In various embodiments of the invention, the ORP characteristics of a biological sample of a subject are measured for purposes of diagnosing, evaluating, monitoring, detecting, or alerting a farmer or animal care-provider of a specific condition within a particular food animal, or within a population of food animals. In such embodiments, the methods can include alerting a farmer of potential disease outbreaks among his/her animals, identify optimum feed diets for optimizing food product quality, and determine optimum husbandry methods for maximizing his/her animals' general health and well-being. The benefits of such methods may result in short-term benefits that immediately affect the animals being tested. However, the benefits may also be gradual and cumulative over a long period of time, resulting in significant improvements to the food product quality produced by future generations of animals, far removed from the first generation of food animal to be tested.

The method of the invention has been found to be useful in the diagnosis, evaluation and monitoring of food animals to assist farmers, breeders, growers, etc. to optimize their animals' particular production characteristics.

As used herein, “production characteristic” refers to a relevant metric of the product being produced by the food animal or a metric of the animal itself. Examples of production characteristics for meat producing food animals include, but are not limited to, taste, flavor, freshness, appearance, color, color stability, fat content, lipid content, protein content, fatty acid content, cholesterol content, susceptibility to oxidation, amino acid content, collagen content, pigment content, fat color, glycogen level, smell, eating quality, juiciness, tenderness, aroma, texture, succulence, pH, R-value, sarcomere length, iron content, water holding capacity, firmness, marbling, cartilage maturity, bone maturity, dark cutting content, cutability, food animal growth rate, liveweight gain, feedlot gain, carcass weight, VIAscan® yield, feed efficiency, adult mortality rate, infection resistance, fertility, pregnancy failures, abortion rate after first trimester, young born dead or die within 24 hours (dystocia), young die after 24 hours of age and weaning, calf-crop weaning percentage (number of calves weaned divided by number of females exposed to bulls), eye health, teeth health, feet health, and leg health.

Examples of production characteristics for egg producing food animals include, but are not limited to, eggs produced per animal per day, shell soundness, shell texture, shell shape, shell color, shell thickness, egg size, relative viscosity of the albumen, presence of foreign matter in the albumen, yolk firmness, yolk color, absence/presence of yolk defects, adult mortality rate, and infection resistance.

Examples of production characteristics for dairy producing food animals include, but are not limited to, bulk tank standard plate count (SPC), somatic cell count (SCC), lab pasteurized count (LPC), coliform count, preliminary incubation count (PIC), butterfat content, protein content, other solids content, rolling herd averages (pounds milk produced, % butterfat, % protein), rolling heard averages (pounds milk produced, % fat, % protein), first lactation metrics (pounds milk produced, % butterfat, % protein), greater than three lactation metrics (pounds milk produced, % butterfat, % protein), infection resistance, milk fever rate, ruminal acidocis rate, adult mortality rate, fertility, pregnancy failures, abortion rate after first trimester, born dead or die within 24 hours (dystocia), die after 24 hours of age and weaning, calf-crop weaning percentage (number of calves weaned divided by number of females exposed to bulls), eye health, teeth health, feet health, leg health, udder health, and trace element status.

In the method of the present invention, ORP provides a simple, flexible, robust and cost-effective metric that can be correlated to one or more production characteristics of choice. An ORP that is significantly high compared to normal reference values may indicate development of less than desirable production characteristics, poor animal health and/or disease. Thus, ORP may alert the farmer to the need for preventive action to improve the production characteristic of the animal, lower mortality rates and/or prevent the spread of disease.

Because a healthy animal is generally more productive, the method of the invention is useful for improving the general health and well-being of food animals by improving husbandry methods. For example, ORP can be used on individual animals who are suffering from, or who are suspected of having, certain diseases and their medications and treatments altered in response to subsequent ORP measurements.

The method of the present invention may be used to monitor and optimize the health and/or food characteristics of individual food animals or a group of food animals. For example, periodic ORP measurements of a subpopulation of a dairy herd may provide insights into the health, well-being, and production output of the herd. This may allow the farmer to make more sweeping changes to husbandry methods of his entire herd, thus improving the quality of the herd's entire milk output. Periodic ORP measurements of a subpopulation may allow the farmer to ascertain the effectiveness of particular husbandry methods, thus enabling informed changes and adjustments and better production results that may affect all of a farm's population in a positive manner.

An aspect of the present invention is a method for evaluating a production characteristic of a food animal comprising measuring the oxidation-reduction potential (ORP) of a sample of the animal and determining if the ORP is significantly different than a reference ORP value. In some embodiments of the present invention, the ORP may be selected from the group consisting of static ORP, transition ORP, steady-state ORP, ORP capacity, capacitance and combinations thereof.

In some embodiments of the present invention, the food animal may be for a food selected from the group consisting of meat, dairy, eggs and combinations thereof. In some embodiments of the present invention, the food animal may be selected from the group consisting of bovine, camelids, caprae (goats), lagomorphs (rabbits), ovis (sheep), swine, cervidae (deer), birds, fish, crustaceans, mollusks, reptiles and combinations thereof. In some embodiments of the present invention, the food animal may be selected from the group consisting of chickens, ducks, geese, pigeons, doves, pheasants, partridge, turkey, emu, swan, ostrich, bison, carabao, cattle, buffalo, yak, llama, camel, goat, rabbits, sheep, swine, fish, crustaceans, mollusks, reptiles and combinations thereof.

In some embodiments of the present invention, the food may be meat and the production characteristic may be selected from the group consisting of taste, flavor, freshness, appearance, color, color stability, fat content, lipid content, protein content, fatty acid content, cholesterol content, susceptibility to oxidation, amino acid content, collagen content, pigment content, fat color, glycogen level, smell, eating quality, juiciness, tenderness, aroma, texture, succulence, pH, R-value, sarcomere length, iron content, water holding capacity, firmness, marbling, cartilage maturity, bone maturity, dark cutting content, cutability, food animal growth rate, liveweight gain, feedlot gain, carcass weight, VIAscan® yield, feed efficiency, adult mortality rate, infection resistance, fertility, pregnancy failures, abortion rate after first trimester, young born dead or die within 24 hours (dystocia), young die after 24 hours of age and weaning, calf-crop weaning percentage (number of calves weaned divided by number of females exposed to bulls), eye health, teeth health, feet health, leg health and combinations thereof.

In some embodiments of the present invention, the food may be eggs and the production characteristic may be selected from the group consisting of eggs produced per animal per day, shell soundness, shell texture, shell shape, shell color, shell thickness, egg size, relative viscosity of the albumen, presence of foreign matter in the albumen, yolk firmness, yolk color, absence/presence of yolk defects, adult mortality rate, infection resistance and combinations thereof.

In some embodiments of the present invention, the food may be a dairy product and the production characteristic may be selected from the group consisting of bulk tank standard plate count (SPC), somatic cell count (SCC), lab pasteurized count (LPC), coliform count, preliminary incubation count (PIC), butterfat content, protein content, other solids content, rolling herd averages (pounds milk produced, % butterfat, % protein), rolling heard averages (pounds milk produced, % fat, % protein), first lactation metrics (pounds milk produced, % butterfat, % protein), greater than three lactation metrics (pounds milk produced, % butterfat, % protein), mortality rate, infection resistance, milk fever rate, ruminal acidocis rate, adult mortality rate, fertility, pregnancy failures, abortion rate after first trimester, born dead or die within 24 hours (dystocia), die after 24 hours of age and weaning, calf-crop weaning percentage (number of calves weaned divided by number of females exposed to bulls), eye health, teeth health, feet health, leg health, udder health, trace element status and combinations thereof.

In some embodiments of the present invention, the sample may be selected from the group consisting of whole blood, blood serum, blood plasma, urine, feces, tissue, muscle, cartilage, bone, milk, vaginal secretions, saliva, sweat, semen, amniotic fluid, cerebrospinal fluid, aqueous humour, vitreous humour, bile, chyle, chyme, gastric juice, lymph, pericardial fluid, peritoneal fluid, pleural fluid, pus, rheum, sputum, synovial fluid, sebum, egg yolk, egg albumen and combinations thereof.

In some embodiments of the present invention, the method for evaluating a production characteristic of a food animal may further comprise adjusting the environment to change the production characteristic of the food animal, wherein the food animal whose production characteristic to be changed may be selected from the group consisting of the food animal, a progeny of the food animal, a cohort of the food animal, a next generation of the food animal and combinations thereof.

In some embodiments of the present invention, adjusting the environment may be selected from the group consisting of administering a treatment to the food animal, changing the dietary regiment of the food animal, altering the living environment of the food animal, and combinations thereof.

In some embodiments of the present invention, adjusting the environment may comprise administering a treatment selected from the group consisting of medications, therapeutics, herbal treatments, ointments, salves, vaccinations, hormones, antibiotics, antioxidants, anti-inflammatories, antivirals, and combinations thereof.

In some embodiments of the present invention, adjusting the environment may comprise changing the dietary regiment selected from the group consisting of food ration delivery methods, hay, grains, protein, vitamin supplements, mineral supplements, free or restricted access to feed, frequency of water provided, volume of water provided per unit time, the water quality, feed composition, daily allowance of protein, the dietary percentage of protein, scratch grains, grains provided, straw provided, grain to straw ratio, presence or absence of fertilizers and/or pesticides in feed, supplements ([for poultry] cracked corn, crushed oyster shells, crushed egg shells, fermented food scraps, flax seeds, grit, antioxidants, feeding limestone, salt, probiotics, crab meal, kelp, fish meal, cultured yeast, and broad-spectrum mineral supplements), and combinations thereof.

In some embodiments of the present invention, adjusting the environment may comprise altering the living environment selected from the group consisting of [for poultry] frequency of spiking male introduction, ratio of hens to roosters, presence/absence of other species of food animals on farm, presence/absence of fencing, presence/absence of hard surface surrounding enclosures, presence/absence of tall brush surrounding enclosures, presence/absence of biosecurity protocols, presence/absence of dedicated service vehicles, number of animals per unit area, number of animal houses, age of animal houses, cleaning procedures, downtime between flocks, visitor access frequency, spent- and dead-animal disposal methods, rodent and insect control measures, food product handling methods, lighting intensity, lighting duration, enclosure temperature, enclosure humidity, [for cattle] access to pasture, access to direct sunlight, ventilation, foggers, misters, sprinklers, cooling, stall surfaces, bedding, stall design, handling methods, loading methods, muster methods, occurrence and/or frequency of pen/mob mixing, method of dispatch, and combinations thereof.

In some embodiments of the present invention, the reference ORP value may be determined based on factors selected from the group consisting of animal species, animal age, geographic location, any one of the above adjusting methods and combinations thereof. In some embodiments of the present invention, the measuring, determining, and adjusting steps may be performed on a single living food animal.

In some embodiments of the present invention, the method for evaluating a production characteristic of a food animal may further comprise dispatching the food animal, wherein the measuring and determining steps are performed on the sample of the butchered food animal. In some embodiments of the present invention, wherein the reference ORP value may be measured on the same food animal sample and the step of measuring the reference ORP value may be performed in a similar manner. In some embodiments of the present invention, the step of measuring the ORP may be performed at least once over the lifespan of the food animal.

In some embodiments of the present invention, the method for evaluating a production characteristic may be performed on a population of food animals, wherein the population may be selected from a group consisting of a flock, a herd, a cohort, a shoal, a brood, a clutch, a bed, a band, a litter, an army, a school, a hedge, a gang, a pod, a brace, a pack, a mob, a team, a stud, a troop, and combinations thereof.

As previously mentioned, in some embodiments the sample may be the food product itself. For example, the sample being measured can be a sample of meat from an animal, a sample of an egg or a sample of milk from an animal. ORP values determined from such samples can be used to determined such things as, for example, taste, freshness, color stability and/or other product characteristics.

In some embodiments, the ORP of a sample of meat from a production animal is measured. Such ORP can be used to determine one or more characteristics of the meat such as, for example, taste, flavor, freshness, appearance, color, color stability, fat content, lipid content, protein content, fatty acid content, cholesterol content, susceptibility to oxidation, amino acid content, collagen content, pigment content, fat color, glycogen level, smell, eating quality, juiciness, tenderness, aroma, texture, succulence, pH, R-value, sarcomere length, iron content, water holding capacity, firmness, marbling, cartilage maturity, bone maturity, dark cutting content and cutability.

Meat color is the single most important factor in a consumer's decision to purchase meat. Consumers typically associate bright, cherry-red meat color with freshness. Variations in meat color come from interactions of compounds in the meat with its environment, and in particular, oxygen. Myoglobin, which is a heme iron containing protein similar to hemoglobin, stores oxygen in muscle cells and is the main protein responsible for giving meat its color. The more myoglobin content meat has the darker red it will appear in color. Myoglobin has three natural colors, depending on its exposure to oxygen and the chemical state of the iron. If no oxygen is present, the myoglobin is in the deoxymyoglobin state and the meat appears purple red in color. When the meat is exposed to oxygen, the oxygen binds to the iron forming oxymyoglobin and the surface of the meat becomes a bright-cherry red color. Both myoglobin and oxymyoglobin have the capacity to lose an electron (oxidation), thereby forming met myoglobin, which renders the meat a brown color. Thus, myoglobin can change from a dark purple color to a bright red color from oxygenation, or to a brown color by loss of electrons. Consequently, the pigments myoglobin, oxymyoglobin and metmyoglobin can be changed from one to another, depending on the conditions under which the meat is stored. Moreover, cooking meat causes formation of denatured metmyoglobin, which cannot be changed into another form of the pigment.

One aspect of the invention is a method of predicting the color stability of a sample of meat, the method comprising determining one or more ORP value(s) of the meat sample and predicting the color stability of the meat from the ORP value. In some embodiments, the ORP may be selected from the group consisting of static ORP, transition ORP, steady-state ORP, ORP capacity, capacitance and combinations thereof. In some embodiments, an increased ORP value indicates decreased color stability over time. In some embodiments, a decreased ORP value indicates increased color stability over time. In some embodiments, the ORP is obtained directly from the meat sample. In some embodiments, a portion of the meat sample is obtained and treated, and the ORP value determined there from. In some embodiments, the ORP value is compared with one or more reference ORP values that have been correlated with color stability over time. In some embodiments, the degree of color stability is used to determine the desirability and/or salability of the meat. In a particular embodiment, the cORP value is measured and the color stability of the meat is determined there from. A higher susceptibility to oxidation will result in less color stability. Consequently, a higher resistance to oxidation (e.g., greater cORP) will slow the process of oxidation, leading to an increase in color stability of the meat. Increased color stability results in higher consumer appeal and thus, increased desirability and salability.

The invention now being generally described will be more readily understood by reference to the following examples, which are included merely for the purposes of illustration of certain aspects of the embodiments of the present invention. The examples are not intended to limit the invention, as one of skill in the art would recognize from the above teachings and the following examples that other techniques and methods can satisfy the claims and can be employed without departing from the scope of the claimed invention.

### EXAMPLES

### Example 1

### Correlation of Myoglobin and Lipid Oxidation with Oxidation Reduction Potential

Immediately after slaughter, antioxidant mechanisms lose their efficiency. Thus, the antioxidant level prior to slaughter can play a significant role in meat quality. Measures of oxidation reduction potential (ORP) provide a snap shot of the current net balance of all oxidants and reductants (sORP) and can provide measures of the antioxidant ORP capacity (cORP), thus it could be used assess the relative levels of oxidative stress prior to slaughter. Protein and lipid oxidation are interrelated by-products of oxidative stress, and provide an insight into the degree of oxidative damage in the post-mortem muscle. The aim of this example was to correlate the myoglobin and lipid oxidation with ORP measures in a sarcoplasm-liposome system.

Sarcoplasm was extracted from 21 d aged beef longissimus steaks. Briefly, 5 g of meat was mixed with 15 mL of 50 mM phosphate buffer at pH 5.6 and homogenized for 30 s using a Polytron tissue homogenizer. The homogenates were centrifuged at 15,000 g for 3 min and the supernatant was used as sarcoplasm. Sarcoplasm was incorporated within a phosphatidylcholine liposome preparation and incubated at 4° C. under a continuous fluorescent lighting for 24 h. at specific time points, and the liposome-sarcoplasm mixture was used to measure thiobarbituric acid reactive substances (TBARS), myoglobin oxidation, sORP, and antioxidant capacity (cORP). Myoglobin oxidation was determined using an integrated sphere spectrophotometer, while sORP and cORP were measured using RedoxSYS analyzer. The experiments were replicated five times (n=5). The data were analyzed and the significance determined at P<0.05. Proc Corr statement was used to determine the correlation between various parameters.

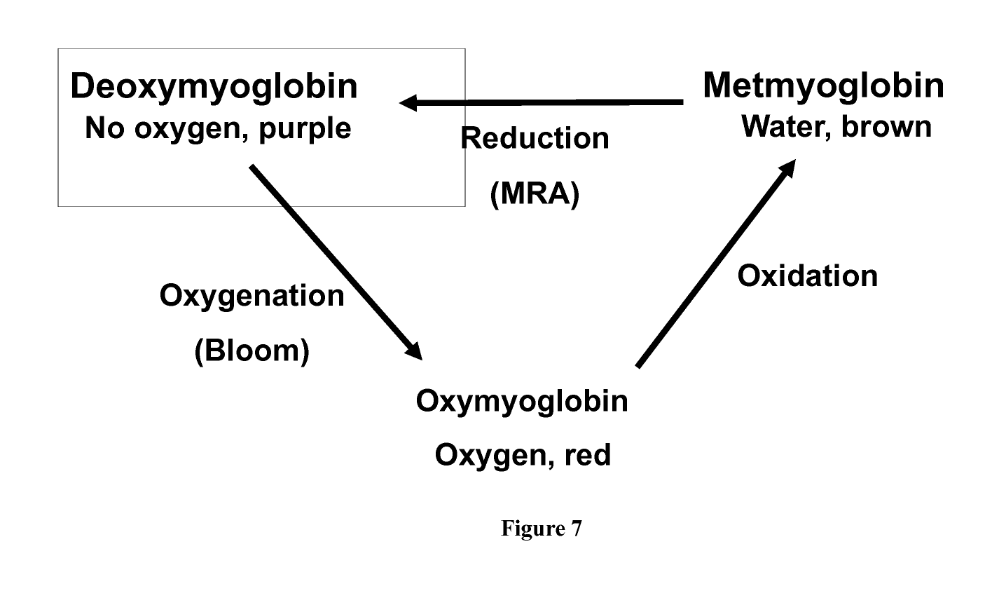

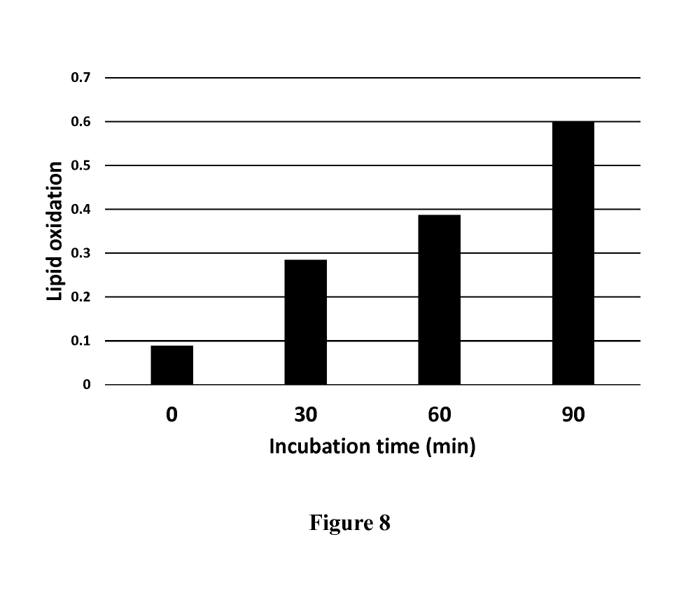

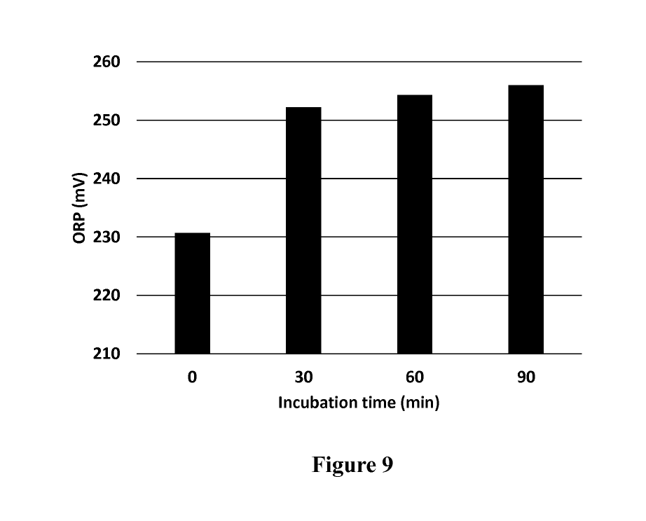

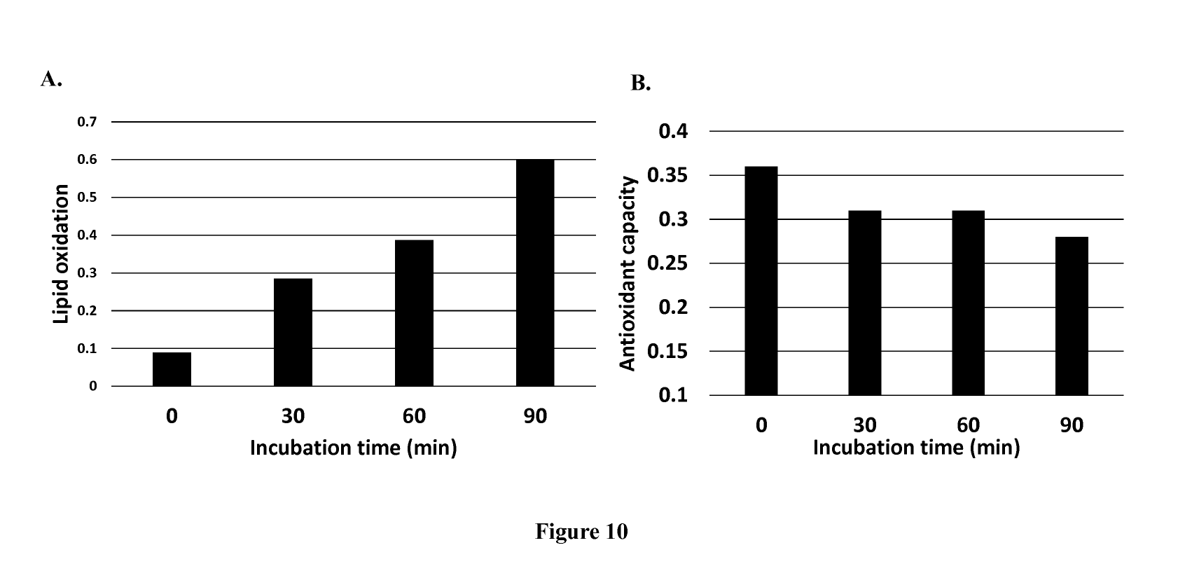

Lipid oxidation, sORP, and metmyoglobin formation of the sarcoplasm-liposome mixture increased (P<0.05) with time (FIGS. 7-9). A greater ORP indicates more oxidative stress. The antioxidant capacity ORP (cORP) of the sarcoplasm-liposome mixture decreased (<0.05) over time (FIG. 10B). Metmyoglobin formation was positively correlated with ORP and lipid peroxidation, and negatively correlated with antioxidant capacity. The results indicate that ORP and antioxidant capacity correlate with lipid peroxidation and metmyoglobin formation in beef.

### Example 2

### Correlation of ORP with Packaging Methods, pH and Metmyoglobin Formation in Beef

Metmyoglobin reducing property of meat is an important inherent biochemical property that influences color stability. The RedoxSYS analyzer is a fast and new proprietary diagnostic system that efficiently determines the ORP properties of biological systems. ORP measures the net balance between total oxidants and total reductants; it is not limited to specific oxidants or reductant families. It has been used previously to demonstrate that multi-trauma patients have higher serum ORP values than healthy controls. The objective of this example was to use RedoxSYS to measure total ORP in beef and correlate the ORP with pH, packaging methods and metmyoglobin formation.

Sarcoplasm was extracted from 21 d aged normal pH and high-pH beef longissimus steaks. The average pH values of normal and high-pH beef were 5.6 and 6.5, respectively. For the second experiment, steaks from normal pH beef longissimus loins were packaged in either PVC or HiOx (80% oxygen and 20% carbon dioxide), and displayed under continuous fluorescent lighting for 5 d. For both experiments, 5 g of meat was mixed with 15 mL of 50 mM phosphate buffer at pH 7.4 and homogenized for 30 s using a Polytron tissue homogenizer. The homogenates were centrifuged at 15,000× g for 3 min and 25 μL of supernatant was used to measure ORP. In addition to ORP measurements, steaks were also used to measure metmyoglobin reducing activity using nitric oxide metmyoglobin reduction method. The experiments were replicated eight times (n=8). The data for the experiment 1 and 2 were analyzed using the Mixed Procedure of SAS and the significance was determined at P<0.05.

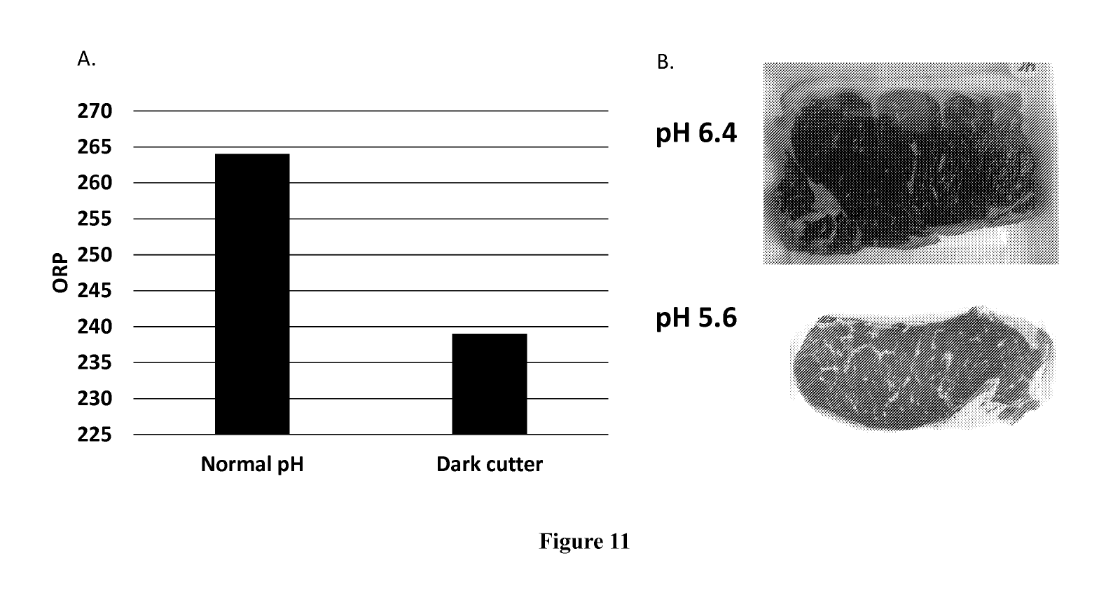

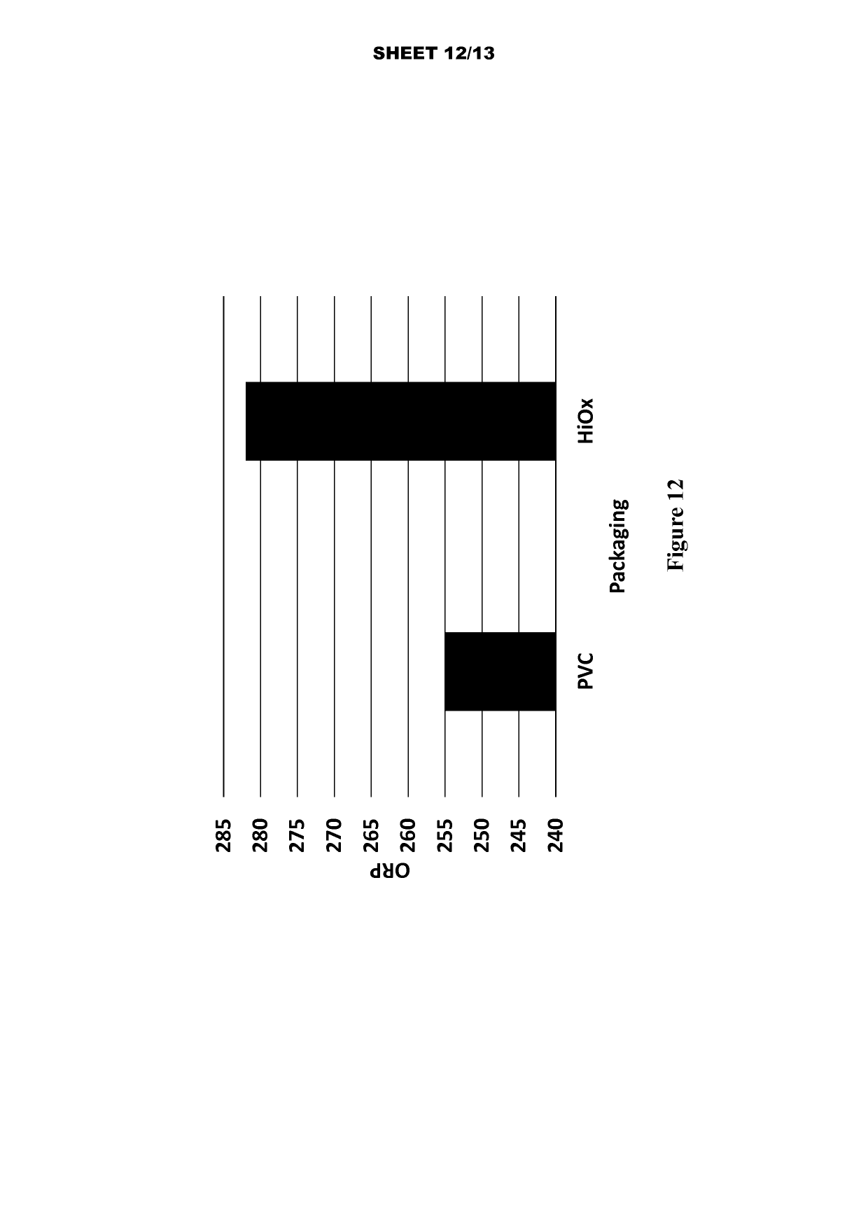

The average reduction potential values of normal and high-pH beef were 262±5 and 239±8 mV, respectively (P<0.05). (FIG. 11A) The greater pH aged beef had significantly lower ORP values and thus was under lower levels of oxidative stress. (FIG. 11A) Metmyoglobin reducing activity was greater (P<0.05) in high-pH beef compared with normal pH beef. (FIG. 11B) Packaging in HiOx conditions increased the oxidative stress compared with PVC packaging (HiOx=282±7 and PVC=255±8 mV; P<0.05) (FIG. 12). The results indicate that ORP can be used to study meat color.

### Example 3

### Effect of Time and Temperature on ORP in Meat

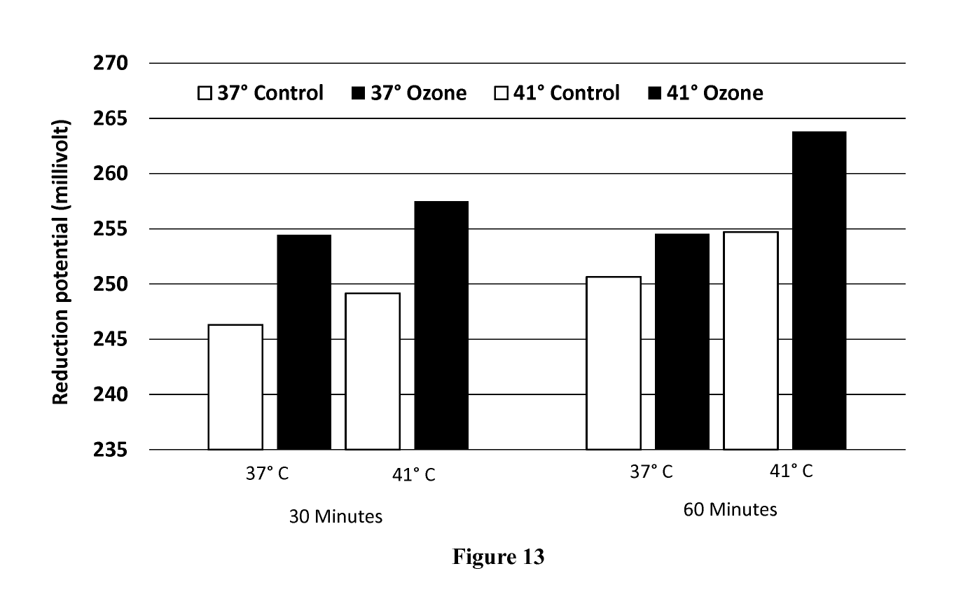

Samples of beef were exposed to ozone and incubated for various times. ORP was measured and compared to meat samples not exposed to ozone. The results of these analyses are shown in FIG. 13. The results show that packaging of meat in the presence of ozone results in an increase in ORP. The results also show that incubation of the meat at higher temperature results in an increased ORP.

What is claimed:

1. A method for evaluating a production characteristic of a food animal comprising:
measuring the oxidation-reduction potential (ORP) of a sample of the animal; and determining if the ORP is significantly different than a reference ORP value.

2. The method of claim 1, wherein the food animal is for a food selected from the group consisting of meat, dairy, eggs and combinations thereof.

3. The method of claim 1, wherein the food animal is selected from the group consisting of bovine, camelids, caprae, lagomorphs, ovis, swine, cervidae, birds, fish, crustaceans, mollusks, reptiles, chickens, ducks, geese, pigeons, doves, pheasants, partridge, turkey, emu, swan, ostrich, bison, carabao, cattle, buffalo, yak, llama, camel, goat, rabbits, sheep, swine, fish, crustaceans, mollusks, reptiles and combinations thereof.

4. The method of claim 1, wherein the production characteristic is selected from the group consisting of taste, flavor, freshness, appearance, color, color stability, fat content, lipid content, protein content, fatty acid content, cholesterol content, susceptibility to oxidation, amino acid content, collagen content, pigment content, fat color, glycogen level, smell, eating quality, juiciness, tenderness, aroma, texture, succulence, pH, R-value, sarcomere length, iron content, water holding capacity, firmness, marbling, cartilage maturity, bone maturity, dark cutting content, cutability, food animal growth rate, liveweight gain, feedlot gain, carcass weight, VIAscan® yield, feed efficiency, adult mortality rate, infection resistance, fertility, pregnancy failures, abortion rate after first trimester, number of young born dead or die within 24 hours, number of young die after 24 hours of age and weaning, calf-crop weaning percentage, eye health, teeth health, feet health, leg health, eggs produced per animal per day, shell soundness, shell texture, shell shape, shell color, shell thickness, egg size, relative viscosity of the albumen, presence of foreign matter in the albumen, yolk firmness, yolk color, absence/presence of yolk defects, adult mortality rate, infection resistance, bulk tank standard plate count, somatic cell count, lab pasteurized count, coliform count, preliminary incubation count, butterfat content, protein content, other solids content, rolling herd averages, rolling heard averages, first lactation metrics, greater than three lactation metrics, mortality rate, infection resistance, milk fever rate, ruminal acidocis rate, adult mortality rate, udder health, trace element status and combinations thereof.

5. The method of claim 1, wherein the sample is selected from the group consisting of whole blood, blood serum, blood plasma, urine, feces, tissue, muscle, cartilage, bone, milk, whey, vaginal secretions, saliva, sweat, semen, amniotic fluid, cerebrospinal fluid, aqueous humour, vitreous humour, bile, chyle, chyme, gastric juice, lymph, pericardial fluid, peritoneal fluid, pleural fluid, pus, rheum, sputum, synovial fluid, sebum, egg yolk, egg albumen and combinations thereof.

6. The method of claim 1, further comprising adjusting the environment of a second food animal to change the production characteristic of the second food animal to be different from the previous production characteristic of the food animal from which the sample was taken, wherein the second food animal is selected from the group consisting of the food animal, a progeny of the food animal, a cohort of the food animal, a next generation of the food animal and combinations thereof.

7. The method of claim 6, wherein adjusting the environment is selected from the group consisting of administering a treatment to the second food animal, changing the dietary regiment of the second food animal, altering the living environment of the second food animal, and combinations thereof.

8. The method of claim 1, wherein the step of measuring the ORP is selected from the group consisting of measuring static ORP and capacity ORP.

9. The method of claim 1, wherein the reference ORP value is determined based on factors selected from the group consisting of animal species, animal age, geographic location, and combinations thereof.

10. A method of determining a characteristic of a food product from a food animal, comprising measuring one or more ORP values of the food product, and determining the characteristic from the measured ORP value.

11. The method of claim 10, wherein the food product is selected from the group consisting of meat, eggs and milk.

12. The method of claim 10, wherein the food animal is is selected from the group consisting of bovine, camelids, caprae, lagomorphs, ovis, swine, cervidae, birds, fish, crustaceans, mollusks, reptiles, chickens, ducks, geese, pigeons, doves, pheasants, partridge, turkey, emu, swan, ostrich, bison, carabao, cattle, buffalo, yak, llama, camel, goat, rabbits, sheep, swine, fish, crustaceans, mollusks, reptiles and combinations thereof.

13. The method of claim 10, wherein the characteristic is selected from the group consisting of taste, flavor, freshness, appearance, color, color stability, fat content, lipid content, protein content, fatty acid content, cholesterol content, susceptibility to oxidation, amino acid content, collagen content, pigment content, fat color, glycogen level, smell, eating quality, juiciness, tenderness, aroma, texture, succulence, pH, R-value, sarcomere length, iron content, water holding capacity, firmness, marbling, cartilage maturity, bone maturity, dark cutting content, cutability, food animal growth rate, liveweight gain, feedlot gain, carcass weight, VIAscan® yield, feed efficiency, adult mortality rate, infection resistance, fertility, pregnancy failures, abortion rate after first trimester, number of young born dead or die within 24 hours, number of young die after 24 hours of age and weaning, calf-crop weaning percentage, eye health, teeth health, feet health, leg health, eggs produced per animal per day, shell soundness, shell texture, shell shape, shell color, shell thickness, egg size, relative viscosity of the albumen, presence of foreign matter in the albumen, yolk firmness, yolk color, absence/presence of yolk defects.

14. The method of claim 10, wherein the step of measuring the ORP is selected from the group consisting of measuring static ORP and capacity ORP.

15. A method of optimizing the health and/or food characteristics of a food animal, or a group of food animals, comprising:
measuring one or more ORP values of a food product from the food animal, or a food product from at least one animal in the group of animals;
determining at least one food characteristic from the measured ORP value; and,
based on the measured ORP value, adjusting the environment of a second food animal, or group of animals, to change the production characteristic of the second food animal, or group of animals, to be different from the previous production characteristic of the food animal from which the sample was taken, wherein the second food animal, or group of animals, is selected from the group consisting of the food animal, a progeny of the food animal, a cohort of the food animal, a next generation of the food animal and combinations thereof.

16. The method of claim 15, wherein the food product is selected from the group consisting of meat, eggs and milk.

17. The method of claim 15, wherein the food animal is is selected from the group consisting of bovine, camelids, caprae, lagomorphs, ovis, swine, cervidae, birds, fish, crustaceans, mollusks, reptiles, chickens, ducks, geese, pigeons, doves, pheasants, partridge, turkey, emu, swan, ostrich, bison, carabao, cattle, buffalo, yak, llama, camel, goat, rabbits, sheep, swine, fish, crustaceans, mollusks, reptiles and combinations thereof.

18. The method of claim 15, wherein the characteristic is selected from the group consisting of taste, flavor, freshness, appearance, color, color stability, fat content, lipid content, protein content, fatty acid content, cholesterol content, susceptibility to oxidation, amino acid content, collagen content, pigment content, fat color, glycogen level, smell, eating quality, juiciness, tenderness, aroma, texture, succulence, pH, R-value, sarcomere length, iron content, water holding capacity, firmness, marbling, cartilage maturity, bone maturity, dark cutting content, cutability, food animal growth rate, liveweight gain, feedlot gain, carcass weight, VIAscan® yield, feed efficiency, adult mortality rate, infection resistance, fertility, pregnancy failures, abortion rate after first trimester, number of young born dead or die within 24 hours, number of young die after 24 hours of age and weaning, calf-crop weaning percentage, eye health, teeth health, feet health, leg health, eggs produced per animal per day, shell soundness, shell texture, shell shape, shell color, shell thickness, egg size, relative viscosity of the albumen, presence of foreign matter in the albumen, yolk firmness, yolk color, absence/presence of yolk defects.

19. The method of claim 15, wherein the step of measuring the ORP is selected from the group consisting of measuring static ORP and capacity ORP.

20. The method of claim 15, wherein adjusting the environment is selected from the group consisting of administering a treatment to the second food animal, changing the dietary regiment of the second food animal, altering the living environment of the second food animal, and combinations thereof.
